(19) United States
(12) Patent Application Publication
(10) Pub. No.: US 2003/0219841 A1
(43) Pub. Date: Nov. 27, 2003

(54) Method and reagent for the detection of apoptotic cells and of a protease activated during apoptosis

(76) Inventor: Georg Fertig, Penzberg (DE)

Correspondence Address: Roche Diagnostics Corporation, 9115 Hague Road, Bldg D, P.O. Box 50457, Indianapolis, IN, 46250-0457, US

(21) Appl. No.: 10/278,702

(22) Filed: Oct. 23, 2002

Related U.S. Application Data
(63) Continuation of application No. 09/306,582, filed on May 6, 1999, now abandoned.

(30) Foreign Application Priority Data
May 7, 1998 (DE) 198 20 328.4

(51) Int. Cl. G01N 33/574; G01N 33/53; G01N 33/537; G01N 33/543

(52) U.S. Cl. 435/7.23; 435/7.92

(57) ABSTRACT

Method and reagent kit for the detection of a protease activated in apoptosis or of apoptotic cells in a biological sample comprising the following steps:

contacting the sample with (a) a binding partner which specifically binds a protease activated in apoptosis but does not block the active centre and is bound or can be bound to a solid phase, (b) a substrate that is specific for the protease and (c) a reaction buffer, and

determining the chromogen or fluorochrome formed in the reaction solution as a measure for the apoptotic cells and/or activated protease contained in the sample.

The invention concerns a method for the detection of apoptosis in eukaryotic cells or of an apoptosis-specific protease as well as a suitable reagent kit for this method of detection.

The so-called programmed cell death is of major importance for the development and functionality of tissues, organs and organisms as a whole (J. Cohen, Advances in Immunology 50 (1991), 55-85) and has been observed in a number of diseases such as automimmune or ischaemic damage, various types of cancer, Alzheimer's and other neurodegenerative phenomena (G. M. Cohen, Biochem. J. 326 (1997), 1-16). Cells which have died in this manner are characterised by DNA fragments associated with histones in the cytoplasm as mononucleosomes and oligonucleosomes. Such a manifestation is also observed in an induced cell death such as one which is triggered for example by ionizing irradiation (Yamada et al., Int. J. Radiat. Biol. 53 (1988), 65) or by certain monoclonal antibodies such as e.g. anti-fas (Yonehara et al., J. Exp. Med. 169 (1989), 1747-1756) or anti-APO-1 (Trauth et al., Science 245 (1989), 301-304). Cytotoxic T cells and natural killer cells also cause such an induced cell death (see e.g. S. Curnow et al., Cancer Immunol. Immunother. 36 (1993), 149-155). However, they also cause an increased permeability of the plasma membrane such as is observed in necrotic phenomena (Krahenbuhl et al., Immunol. Today 12 (1991), 389-402). The described type of programmed or induced cell death is denoted apoptosis. Apoptosis is characterized by vesicular protuberances of the plasma membrane, condensation of the chromatin and activation of an endogenous endonuclease. In contrast to necrosis, apoptosis is an active process of the eukaryotic cell. The calcium- and magnesium-dependent endonuclease activated in apoptosis cleaves the DNA double strand into mononucleosomes and oligonucleosomes in the readily accessible linker regions between the nucleosomes. In contrast the DNA in the nucleosomes is closely associated with certain core histones and is therefore protected from cleavage by the endonuclease (Burgoyne et al., Biochem. J. 143 (1974), 67; Stach et al., J. Neurochem. 33 (1979), 257). Therefore the “DNA ladder”—a pattern of DNA fragments with a length of ca. 180 base pairs or a multiple thereof (Wyllie, Nature 284 (1980), 555-556; J. Cohen, Advances in Immunology 50 (1991), 55-85)—that is typical for apoptotic cells is detectable after extraction of the DNA and separation in an agarose gel. The plasma membrane of the cells remains intact in this early stage of apoptosis. In this manner mononucleosomes and oligonucleosomes and other high molecular weight components accumulate in the cytoplasm of the dying cell (Duke et al., Lymphokine Research 5 (1986), 289-299).

Although a number of molecules have been identified in recent years which play a role in the biochemical development of apoptotic cells and there has also been an increasing recognition among experts of the fundamental importance of apoptosis for biological processes which extend from embryogenesis to the development of the immune system, the mechanism for the development of apoptosis still remains largely unclarified (M. Tewari et al., Cell 81 (1995), 801-809). However, recent evidence indicates that certain proteases which belong to the family of cysteine proteases, the so-called “CASPASES” play a key role in the development of apoptosis (D. W. Nicholson and N. A. Thornberry, TIBS 22 (1997), 299-306). In particular it was shown that the zymogen caspase 3 (cysteinyl-aspartic acid protease, CPP-32, apopain, yama) is present in an active form during the development of apoptosis (D. W. Nicholson et al., Nature 376 (1995), 37-43).

Test procedures for the detection of apoptosis or apoptosis-specific proteases have been described and are based essentially on the cleavage of an appropriate peptide substrate and detection of a fluorescent or calorimetric component that is released in this process (e.g. D. W. Nicholson et al., Nature 376 (1995), 37-43). A disadvantage of the present test procedures for the detection of apoptosis-specific proteases is, however, that other proteases that are involved in the development of apoptosis are also analysed which also cleave the peptide substrate and hence they do not have the required specificity.

The object of the invention was therefore to provide a simple and specific test procedure for the detection of apoptosis especially in the early development phase of apoptosis.

This object is achieved by a method for the detection of apoptosis-specific proteases which is characterized in that

a lysate containing the cell population to be examined is incubated with a binding partner which specifically binds a protease activated during apoptosis but does not block the active centre of the protease,

is contacted with a peptide substrate that is specific for the protease and is coupled to a detectable group and

with a reaction buffer and

the detectable group released from the substrate is determined.

In the method according to the invention the binding partner can either be bound to a solid phase such as a microtitre plate or a latex particle before contact with the sample or the lysate or it is coupled to an appropriate solid phase after incubation with the protease substrate. Cysteinyl-aspartic acid proteases such as for example caspase 3 or caspase 8 are especially suitable proteases according to the invention that are activated during apoptosis.

It has surprisingly turned out that apoptotic cells i.e. cells which have died due to programmed or induced cell death already generate a measurement signal after about 1.5 to 2 hours incubation and when there are less than about 1000 apoptotic cells without having to pre-label the cell population under examination. As a result it is also possible to examine non-proliferating cells in vitro. Furthermore the method according to the invention allows a simple and rapid quantitative determination of the extent of apoptosis in the sample to be examined (cell population) and moreover is extremely sensitive.

In order to release the protease(s) activated in apoptosis from the cytoplasm of apoptotic cells, the cell population to be examined is incubated with a suitable lysis buffer which essentially contains an anionic or non-ionic detergent such as Triton, Tween or Nonidet P40 at a concentration of about 0.1 to 2.0% (w/v) and a substance buffering in a pH range of about 5.5 to 9.0, preferably about 7 to 9. For example phosphate, Tris-HCl and Hepes are suitable. The concentration of the buffer is about 1 to 200 mM, preferably ca. 10 to 80 mM, particularly preferably about 50 mM. In addition a substance reducing sulfite groups can be optionally added to the lysis buffer such as dithiothreitol (DTT) or dithioerythritol at a concentration of about 0.1 to 100 μM and/or sodium chloride to ensure a physiological salt concentration.

Incubation with the lysis buffer is carried out for a period of about one to five minutes at temperatures of about 0 to 4° C. Insoluble cell components are removed by centrifugation. The lysate obtained in this manner is added to an appropriate binding partner which is preferably a monoclonal or polyclonal antibody. According to the invention monoclonal antibodies directed against a cysteinyl-aspartic acid protease or an immunologically active fragment thereof are particularly preferred. It has proven to be particularly suitable to use antibodies which have been obtained using a ca. 25 kDa large protein fragment (e.g. of the human CPP-32 (amino acids 1 to 219) or MACH protease) as an immunogen and injecting three times preferably at intervals of ca. 10 days (e.g. clone 19). The clones obtained in this manner are particularly suitable for the method according to the invention after optional chromatographic purification by methods known to a person skilled in the art. Alternatively suitable antibodies can be obtained commercially from appropriate vendors such as the Transduction Laboratories Company, Lexington, Ky. (USA).

The peptide substrate for the protease coupled to a detectable group is for example a peptide composed of two to about 20 amino acids coupled to a chromogenic or fluorescent group. The peptide substrate preferably contains at least 10 amino acids, and it is particularly preferably a tripeptide to octapeptide. In addition cyclic peptide substrates with an integrated cleavage sequence can also be used for the method according to the invention in which case the cleaved peptide complements an enzyme to form its active form. Preferred peptide substrates have in particular peptide fragments of the following amino acid sequences: Trp(Leu)-Glu-His-Asp, Asp-Glu-Val(His)-Asp or one of the following three sequences Val(Leu)-Glu-His(Thr)-Asp. In addition the peptide substrates can be acetylated and preferably contain the detectable group on a terminal, non-acetylated amino acid. Suitable detectable, i.e. chromogenic or fluorescent groups, are preferably coumarin, nitroanilide or naphthylamide derivatives. In particular the following substrates have proven to be suitable according to the invention: acetyl-Asp-Glu-Val-Asp-7-amido-4-trifluoromethyl-coumarin (Ac-DEVD-AFC), acetyl-Asp-Glu-Val-Asp-7-amido-4-methylcoumarin (Ac-DEVD-AMC), (7-methoxycoumarin-4-yl)acetyl-Asp-Glu-Val-Asp-Ala-Pro-Lys(2,4-dinitrophenyl)OH (Mca), (4-(4-dimethylaminophenylazo)benzoyl-Asp-Glu-Val-Asp-5-[(2-aminoethyl)amino]-naphthalene-1-sulfonic acid or rhodamine-110-DEVD derivatives.

The reaction between the sample containing the protease, the binding partner or antibody and the protease substrate can either take place before or after the binding partner or antibody has been bound to the solid phase. Buffer solutions used for the incubation essentially contain a substance buffering in a pH range of ca. 5.5 to 9.0 at a concentration of 1 to 200 mM as well as a substance reducing sulfite groups such as DTT or dithioerythritol at a concentration of about 0.1 to 100 μM, preferably of about 5 to 60 μM and optionally EDTA and/or EGTA at a concentration of ca. 0.1 to 1.0 mM. In addition the buffer can also contain physiological saline and/or a magnesium salt preferably about 1 mM. An additional preferred embodiment of the invention is when the buffer used to lyse the cells and the reaction buffer are identical and contain ca. 0.1 to 1% (w/v) of an appropriate detergent and about 5 to 60 μM of a substance reducing sulfite groups.

Depending on the number of apoptotic cells in the respective sample, the incubation can be carried out at a temperature of ca. 37° C. and pH 7.8 for a period of about 30 minutes to twelve hours. In most cases a reaction period of 1.5 to 5 hours has proven to be adequate.

The procedure described in example 1 or a parallel culture of the cell population to be examined in which no apoptosis has been induced is used as a negative control. The signal measured without the cell lysate yields the blank value for the determination and is subtracted from the measured values. Those measured values for groups that are detectable in the reaction mixture are rated as positive for the presence of apoptosis which are reproducibly at least two-fold above the measured value without induction. In this case a culture of cells of the same cell type and of the same origin is used as a parallel culture of the cell population to be examined in which no apoptosis has been induced where it can be assumed that no apoptosis has been induced on the basis of the previous history of the cells that are used and the culture conditions. If it is intended to determine an apoptosis that has been induced in vitro, the cell population to be examined is divided into two parallel preparations and then preincubated in the presence and absence of the agent inducing apoptosis (e.g. camptothecin).

In a preferred embodiment of the method according to the invention the binding partner is biotinylated in order to bind it to a solid phase coated with streptavidin before or after the incubation.

Hence, with the aid of the method according to the invention it is possible to determine in a simple manner the cytotoxicity of cells or compounds which induce apoptosis as well as proteases specifically induced by apoptosis.

The invention therefore in addition concerns the use of the method according to the invention to determine the cytotoxic effect or activity of cytotoxic cells, natural killer cells, ionizing radiation or chemical substances such as e.g. monoclonal antibodies, hormones (e.g. glucocorticoids) or toxins (e.g. dioxins). The cytotoxic effect or activity is given in this case by the extent of apoptosis determined by means of the method according to the invention in the cell population used as target cells.

The invention also concerns a reagent kit for the detection of apoptotic cells or a protease activated in apoptosis containing a binding partner which specifically binds a protease activated in apoptosis without blocking the active centre of the protease, a substrate specific for the protease and a suitable reaction buffer. The binding partner (caspase 3 in the example) can be present coupled to a solid phase or only be coupled to such a solid phase after the incubation. Preferred embodiments are when the binding partner is a monoclonal or polyclonal antibody which is directed against an approximately 25 kDa protein fragment of the human CPP-32 and/or a fluorescent-labelled peptide substrate such as acetyl-Asp-Glu-Val-Asp-7-amido-4-trifluoromethyl-coumarin.

Furthermore the reagent kit according to the invention has proven to be suitable for finding substances which are able to inhibit proteases activated in the apoptosis state. In addition substances can be found which have an amplifying i.e. inducing effect on proteases activated during apoptosis. Such inhibiting or inducing substances can be used as active substances such as drugs. For example substances which cause a 90% or greater inhibition of proteases that are activated in the apoptotic state can halt the death of apoptotic cells or at least delay their extinction. Such inhibiting substances that have proven to be suitable are for example peptide derivatives such as acetylasparaginyl-glutaminyl-valinyl-aspartic acid aldehyde (Ac-DEVD-CHO) or benzyloxycarbonyl-valinyl-alaninyl-DL-aspartic acid-fluoromethyl ketone (Z-VAD-fmk). In addition substances that are able to induce proteases that are activated in the apoptotic state are valuable tools for the isolation of specific proteases. In this case substances have proven to be suitable which can amplify the activity of such proteases by at least 1.5-fold.

Proteases activated in the apoptotic state are in particular cysteine or cysteinyl-aspartic acid proteases.

### FIGURE LEGEND

FIG. 1 shows the activation of caspase 3 by incubation of human U937 cells with various concentrations of camptothecin compared to a known test procedure (annexin V-binding).

The invention is elucidated further by the following examples.

### EXAMPLE 1

### Protease Activation/Camptothecin

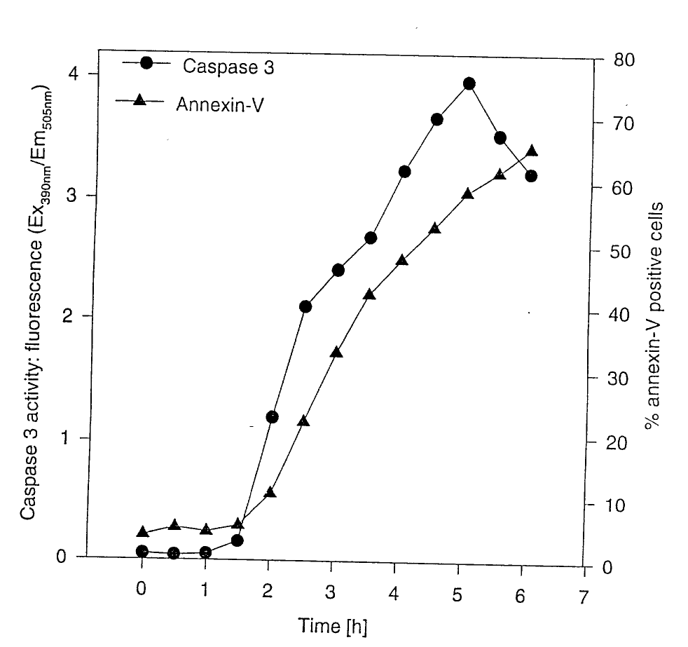

In order to induce apoptosis in a cellular system, human cells (U937; human histiolytic lymphoma of a Caucasian) were treated with camptothecin (Topoisomerase I inhibitor, chemotherapeutic agent). When 5×10⁵ cells/ml are incubated with 4 μg/ml camptothecin (CAM) for various time periods, an increase of protease activity is already detected after 2 h. In order to detect the protease activated by apoptosis (caspase 3), ca. 10⁶ cells were withdrawn from the treated cell culture and lysed in lysis buffer (50 mM NaPO₄, 10 mM NaCl, 1 mM MgCl₂, 0.25% (w/v) Nonidet P40, 0.3 mM EDTA; pH 7.8) on ice. The lysate is then contacted with an antibody directed against caspase 3 which is coupled to a solid phase and binds caspase 3 from the lysate for the specific caspase 3 analysis. After removing the supernatant (contains all unspecific components and enzymes), a substrate (50 μM Ac-DEVD-AFC) is added and its caspase 3 mediated substrate conversion is measured by the formation of the fluorescent coumarin cleavage product. After subtraction of the blank value (self fluorescence of the substrate without lysate addition), the kinetic course of the caspase 3 activity can be shown graphically (FIG. 1).

### EXAMPLE 2

### Colour/Fluorescent Substrate Without a Specific Binding Partner

An unspecific caspase assay (Clonetech) was used as a function test for the cellular model (U937/CAM) which is able to analyse lysates but does not allow a specific capturing of the caspase 3. As in example 1 the substrate used in Ac-DEVD-AFC, and a colorimetric substrate (Ac-DEVD-pNA) was used in parallel. These mixtures are unspecific since each DEVD substrate is cleaved by caspase 3 as well as by caspase 1, casp 2 and casp 7.

The evaluation shows that the apoptosis model functions in principle and that the calorimetric as well as the fluorescent substrate are cleaved during apoptosis induction.

### EXAMPLE 3

### Antibody Selection

Various antibodies recognizing caspase 3 were immobilized on a solid phase (MTP) and thus caspase 3 (activated and non-activated enzyme) was bound from the lysate. However, after addition of the fluorescent substrate (50 μM AMC), a positive signal was only found when using an antibody (e.g. clone 19) according to the invention. The three other antibodies bind caspase 3 but do not generate a significant signal i.e. protease activity cannot be determined.

TABLE 1

| MAB<CPP-32> | Clone 19 | PAB | Ab-1 | Ab-2 |
|---|---|---|---|---|
| −CAM | 2.38 | 2.59 | 2.34 | 2.214 |
| +CAM | 6.65 | 2.88 | 2.31 | 2.344 |
| factor | 2.79 | 1.11 | 0.99 | 1.06 |

### EXAMPLE 4

### Selection of the Optimal Fluorescent Substrate

Three fluorescent substrates were tested. They are acetyl-Asp-Glu-Val-Asp-7-amido-4-trifluoromethyl-coumarin (AFC), acetyl-Asp-Glu-Val-Asp-7-amido-4-methyl-coumarin (AMC) and (7-methoxycoumarin-4-yl)acetyl-Asp-Glu-Val-Asp-Ala-Pro-Lys(2,4-dinitrophenyl)OH (Mca). The three substrates were compared with one another using the assessment criteria i) background ii) signal level and iii) signal ratio from +/− induction. It turned out that although the AMC derivative generated the highest signals, the self-fluorescence of the substrate without lysate addition is relatively high. In contrast the Mca substrate yielded a somewhat better ratio but low signal levels. In contrast an optimal ratio with a very good signal yield was obtained with the AFC substrate.

### EXAMPLE 5

### Optimization of the Incubation Buffer

In order to provide an optimal substrate buffer which can also be used to prepare the cell lysate, various buffer compositions were tested. The assessment showed that the signal levels were extremely dependent on the presence of DTT. Furthermore the optimal ratio and signal yield was found in buffer composition 2 of the tested solutions. In individual cases it is possible to omit EDTA and/or EGTA; however, the presence of EDTA or EGTA has clear advantages when DTT is not used (EDTA and EGTA are only present in buffers 2 and 3 and these are the only buffer combinations which generate a positive signal without DTT). The pH is preferably neutral to weakly alkaline and a broad alkaline pH range still gives acceptable results.

TABLE 2
Buffer solutions 1 to 12 that were used

| 1 | 2 | 3 | 4 | 5 | 6 |
|---|---|---|---|---|---|
| 25 mM Hepes | 50 mM NaPO4 | 50 mM NaPO4 | 100 mM Hepes | PBS | PBS |
| 100 mM NaCl | 10 mM NaCl | 10 mM NaCl | 2 mM MgCl2 | pH 7.4 | 0.1% Triton- |
| 2 mM MgCl2 | 1 mM MgCl2 | 2 mM MgCl2 | 0.1% CHAPS | | X-100 |
| 0.1% Triton- | 0.25% NP-40 | 0.25% NP-40 | 10% sucrose | | pH 7.4 |
| X-100 | 0.3 mM EDTA | 0.3 mM EDTA | 0.1% ovalbumin | | |
| pH 7.5 | 0.3 mM EGTA | 0.3 mM EGTA | pH 7.5 | | |
| | pH 7.8 | pH 7.8 | | | |

| 7 | 8 | 9 | 10 | 11 | 12 |
|---|---|---|---|---|---|
| PBS | 50 mM Hepes | 50 mM Tris | 50 mM Tris-HCl | 50 mM Hepes | 50 mM Tris- |
| 2 mM MgCl2 | 2 mM MgCl2 | 2 mM MgCl2 | 2 mM MgCl2 | 2 mM MgCl2 | 2 mM MgCl2 |
| 0.1% Triton- | 0.1% Triton- | 0.1% Triton- | 0.1% Triton- | 0.1% Triton- | 0.1% Triton- |
| X-100 | X-100 | X-100 | X-100 | X-100 | X-100 |
| pH 7.4 | pH 8.0 | pH 8.0 | pH 7.0 | pH 6.0 | pH 6.0 |

Procedure:

MTP was coated with 2 μg/ml anti-CPP32 (clone 19) (e.g. containing carbonate buffer).

Lysates of untreated and apoptosis-induced cells were added by pipette.

Binding of CPP32 (caspase 3).

Washing.

Addition of fluorescent substrates (AFC) in the respective incubation buffer.

Measurement of FU after 2 h.

TABLE 3
Buffer solutions 1 to 12 with and without addition of DTT

| | | | | | | | | | | | | |
|---|---|---|---|---|---|---|---|---|---|---|---|---|
| buffer | 1 | 2 | 3 | 4 | 5 | 6 | 7 | 8 | 9 | 10 | 11 | 12 |

with 10 μM DTT

| | | | | | | | | | | | | |
|---|---|---|---|---|---|---|---|---|---|---|---|---|
| −CAM | 0.201 | 0.17 | 0.173 | 0.14 | 0.166 | 0.129 | 0.145 | 0.145 | 0.169 | 0.203 | 0.166 | 0.209 |
| +CAM | 2.349 | 2.907 | 2.499 | 1.715 | 2.059 | 2.174 | 2.234 | 0.969 | 1.656 | 1.492 | 0.494 | 0.994 |
| factor | 11.69 | 17.10 | 14.45 | 12.25 | 12.40 | 16.85 | 15.41 | 6.68 | 9.80 | 7.35 | 2.98 | 4.76 |

without 10 μM DTT

| | | | | | | | | | | | | |
|---|---|---|---|---|---|---|---|---|---|---|---|---|
| −CAM | 0.152 | 0.134 | 0.131 | 0.16 | 0.135 | 0.135 | 0.136 | 0.127 | 0.137 | 0.159 | 0.145 | 0.15 |
| +CAM | 0.185 | 0.337 | 0.334 | 0.145 | 0.12 | 0.015 | 0.125 | 0.121 | 0.113 | 0.184 | 0.14 | 0.181 |
| factor | 1.22 | 2.51 | 2.55 | 0.91 | 0.89 | 0.11 | 0.92 | 0.95 | 0.82 | 1.16 | 0.97 | 1.21 |

Result:

1) Hardly any signal is generated without DTT in a lysate containing caspase 3.

2) The concentration of DTT can be between 1 and 100 μM.

3) The evaluation shows that buffer composition 2 produces the highest signal (FU) and the largest factor (apoptotic to non-apoptotic lysate)

### EXAMPLE 6

### Antibody Production

In order to produce monoclonal antibodies against caspase 3, a 24.7 kDa protein fragment (amino acids 1-219 of human CPP-32) was used as an immunogen. For the immunization, mice (e.g. Balb/c) were injected three times with the immunogen (ca. 50 μg peptide) at intervals of 10 days. After about 50 weeks (depending on the immune response of the serum), the spleen cells of the serum-positive mice are fused with myeloma cells (e.g. P3-X63-Ag8.653). The hybridoma supernatants are screened for a positive immunoreaction (immuno-histochemistry, Western blot). Positive hybridomas are cloned in a limiting dilution process. Monoclonal antibodies are purified by means of chromatographic methods and stored in 20 mM sodium phosphate pH 7.5, 50% glycerol, 150 mM NaCl, 1 mg/ml BSA and 1.5 mM NaN₃.

1. A method for the detection in a biological sample of a cysteinyl aspartic acid protease that exhibits increased activity during apoptosis, said method comprising the following steps:
a. contacting said sample with a binding partner selected from the group consisting of antibodies against said cysteinyl aspartic acid protease and immunologically active fragments thereof which specifically binds said protease but does not block the active centre of said protease and which is bound or capable of being bound to a solid phase, a substrate that is specific for said protease coupled to a detectable group and cleavable by said protease, and a reaction buffer,
b. measuring said detectable group cleaved from said substrate by said protease as a measure of said cysteinyl aspartic acid protease contained in said sample, and
c. correlating the measurement of said detectable group with the activity of said cysteinyl aspartic acid protease.

2. The method of claim 1, wherein said protease is caspase-3.

3. The method of claim 1, wherein said binding partner is a monoclonal or polyclonal antibody.

4. The method of claim 1, wherein said substrate is selected from the group consisting of a coumarin-peptide derivative, para-nitroanilide and a naphthylamide-peptide derivative.

5. The method of claim 1, wherein said substrate is selected from the group consisting of acetyl-Asp-Glu-Val-Asp-7-amido-4-trifluoromethyl-coumarin, acetyl-Asp-Glu-Val-Asp-7-amido-4-methylcoumarin, and (7-methoxycoumarin-4-yl) acetyl-Asp-Glu-Val-Asp-Ala-Pro-Lys (2,4-dinitrophenyl) OH.

6. The method of claim 1, wherein said reaction buffer has a pH value of about 5.5 to 9.0 and contains a detergent and a substance that reduces sulfide groups.

7. The method of claim 1, wherein said sample comprises at least 1000 apoptotic cells.

8. The method of claim 1, wherein said steps are carried out at about 37° C. for a period of about 30 minutes to twelve hours.

9. The method of claim 8, wherein said period is 1.5 to 5 hours.

10. A reagent kit for the detection in a biological sample of a cysteinyl aspartic acid protease that exhibits increased activity during apoptosis comprising:
a. a binding partner selected from the group consisting of antibodies against said cysteinyl aspartic acid protease and immunologically active fragments thereof which specifically binds said protease but does not block the active centre of said protease and which is bound or capable of being bound to a solid phase,
b. a specific substrate for said protease coupled to a detectable group and cleavable by said protease, and
c. a reaction buffer.

11. The reagent kit of claim 10, wherein said binding partner is a monoclonal or polyclonal antibody.

12. The reagent kit of claim 10, wherein said binding partner is an antibody capable of being obtained by using a fragment of human caspase 3 protease as an immunogen.

13. The reagent kit of claim 10, wherein said substrate is acetyl-Asp-Glu-Val-Asp-7-amido-4-trifluoromethyl-coumarin.


SEQUENCE LISTING (8 sequences)

SEQ ID NO: 1 - protein, 4 aa, Artificial Sequence
  This is a peptide region of a substrate
XEHD

SEQ ID NO: 2 - protein, 4 aa, Artificial Sequence
  This is a peptide region of a substrate
DEXD

SEQ ID NO: 3 - protein, 4 aa, Artificial Sequence
  This is a peptide region of a substrate
XEXD

SEQ ID NO: 4 - protein, 4 aa, Artificial Sequence
  This is a peptide region of a substrate
DEVD

SEQ ID NO: 5 - protein, 4 aa, Artificial Sequence
  This is a peptide region of a substrate
DEVD

SEQ ID NO: 6 - protein, 7 aa, Artificial Sequence
  This is a peptide region of a substrate
DEVDAPK

SEQ ID NO: 7 - protein, 4 aa, Artificial Sequence
  This is a peptide region of a substrate
DEVD

SEQ ID NO: 8 - protein, 4 aa, Artificial Sequence
  This is a peptide region of a substrate
DEVD